(19) United States
(12) Patent Application Publication
(10) Pub. No.: US 2005/0004482 A1
(43) Pub. Date: Jan. 6, 2005

(54) Amplified system for determining parameters of a patient

(76) Inventor: Budimir Drakulic, Los Angeles, CA (US)

Correspondence Address: ELLSWORTH R. ROSTON, ESQ.;FULWIDER PATTON LEE & UTECHT, LLP, HOWARD HUGHES CENTER, 6060 CENTER DRIVE, TENTH FL., LOS ANGELES, CA, 90045, US

(21) Appl. No.: 10/611,696

(22) Filed: Jul. 1, 2003

(51) Int. Cl. A61B 5/04

(52) U.S. Cl. 600/509

(57) ABSTRACT

An electrode is attached at a selective position to a patient's body to provide signals representative of the patient's parameters (e.g., electrocardiogram) at that position. The electrode signal may be in microvolts or millivolts. Depending upon the characteristics of the patient's skin, the electrode impedance may vary to approximately 200 kilohms. The electrode signals pass to an amplifier having an input impedance (e.g., 10¹⁵ ohms) approaching infinity and a low output impedance The amplifier impedances insure that the electrode signal will pass through the amplifier without loss in signal strength and without change in signal characteristics. A low pass filter connected to the amplifier output eliminates noise and passes signals at low frequencies (e.g., 1 kilohertz). The filter and the amplifier are disposed on a printed circuit board with the amplifier physically and electrically isolated from the filter. Another low pass filter may be connected to the input of the amplifier.

This invention relates to a system for amplifying signals from electrodes attached to a patient's skin without any loss in signal strength and without any change in signal characteristics.

### BACKGROUND OF THE INVENTION

Measurements are provided in a patient of the functioning of various organs in a patient's body. For example, measurements are made of the functioning of the patient's heart, the patient's brain and the patient's stomach and intestinal tract. These measurements are generally made by applying an electrode to the skin of the patient at the appropriate position or positions on the patient's body.

The measurements of the functioning of different organs in the patient's body involve different frequency ranges. For example, measurements of the patient's heart occur in a range of DC to approximately two hundred and fifty hertz (250 Hz); measurements of the patient's brain occur in a range of DC to approximately one hundred and fifty hertz (150 Hz); and measurements of the functioning of the patient's stomach and intestinal tract occur in a range of DC to approximately one hertz (1 Hz).

The measurement of the functioning of different organs in the patient's body involve signals of miniscule amplitude. For example, the range of voltages produced at an electrode attached to the patient's skin for a measurement of the patient's heart is in a range of approximately one half of a millivolt (0.5 mV) to approximately four millivolts (4 mV); a range of voltages produced at an electrode attached to the patient's skin for a measurement of the patient's brain is in a range of approximately five microvolts (5 μV) to approximately three hundred microvolts (300 μV); and a range of voltages for the functioning of the patient's stomach and intestines is in a range of approximately ten microvolts (10 μV) to approximately one thousand microvolts (1000 μV).

When an electrode is attached to a patient's skin to measure the functioning of an organ such as the patient's brain, heart or stomach or intestinal tract, the voltage generated from the organ has to penetrate from the organ through the patient's skin to the electrode. This is probably one reason why the voltage produced at the electrode is in the range of millivolts from the heart and in the range of microvolts from the brain and the stomach and intestinal tract.

The skin has many layers. The greater the number of layers that the voltage has to penetrate in the patient's skin, the greater is the impedance that the skin presents to the voltage generated by the organ whose function is being measured. The problem of high impedances is compounded if the patient's skin is not clean when the measurement is being made. Thus, the impedance presented by the patient's skin may vary from a low impedance to an impedance of approximately two hundred thousand (200,000) ohms.

In view of the different parameters (e.g. signal frequency, voltage range and skin impedance) provided for measurements of different organs in a patient's body, special instruments have been provided to measure the functions of the different organs in the patient's body. For example, instruments for measuring the functioning of a patient's heart are not used to measure the functioning of a patient's brain or a patient's stomach or intestinal tract. Separate instruments have been used to measure the functioning of different organs in a patient's body even though the need or at least the desirability of providing a universal instrument capable of measuring the functioning of different organs in the patient's body has been recognized for some time.

Applicant filed application Ser. No. 10/293,105 (attorney's file RECOM-61830) in the U.S. PTO on Nov. 13, 2002 for a System For, and Method of, Acquiring Physiological Signals of a Patient and has assigned this application Ser. No. 10/293,105 to the assignee of record of this application. application Ser. No. 10/293,105 discloses and claims a system including a plurality of channels each of which has properties of producing signals indicating the functionality of any one of a number of different organs in a patient's body. As disclosed in application Ser. No. 10/293,105, each channel is adapted to be coupled to any one of a number of organs in the patient's body. Each channel includes an amplifier which is operable to produce signals representing the functionality of any one of the organs to which the channel is coupled.

### BRIEF DESCRIPTION OF A PREFERRED EMBODIMENT OF THE INVENTION

This invention provides an amplifier system which provides an amplification of the signals from any one of a plurality of organs in a patient's body regardless of the organ to which the amplifier system is coupled. The amplifier system includes an amplifier which is operative to amplify the signals from any selected one of the organs in the patient's body without any loss in the signal strength and without any changes in the characteristics of the signals.

In accordance with a preferred embodiment of the invention, an electrode is attached at a selective position to a patent's body to provide signals representative of the patient's parameters (e.g., electrocardiogram) at this position. The electrode signal may be in the order of microvolts or millivolts. Depending upon the characteristics of the patient's skin, the electrode-skin impedances may vary to approximately 200 kilohms. The electrode signals pass to an amplifier having an input impedance (e.g., 10¹⁵ ohms) approaching infinity and a low output impedance (e.g. 50 ohms). The amplifier impedances ensure that the electrode signal will pass through the amplifier without loss in signal strength and change in signal characteristics. A low pass filter connected to the amplifier input eliminates noise and passes signals at low frequencies (e.g., 1 kilohertz maximum).

### BRIEF DESCRIPTION OF THE DRAWINGS

FIG. 1 is a circuit diagram, substantially in block form, of an amplifier system, including a pair of amplifiers and a pair of electrodes, constituting a preferred embodiment of the invention;

FIG. 2 is a circuit diagram of one of the amplifiers included in the amplifier system shown in FIG. 1;

FIG. 3 is a schematic perspective view of the different layers in a patient's skin;

FIG. 4 is a simplified elevational view of an electrode, a patient's skin (on a simplified basis) and a gel for facilitating the coupling between the electrode and the patient's skin and also shows the impedance network formed by the electrode, the gel and the patient's skin;

FIG. 5 is a schematic perspective view showing the attachment of an electrode in FIG. 1 to a patient's skin to provide signals from organs (e.g., heart) in the patient's body for introduction to the amplifier system also shown in FIGS. 1 and 2; and

FIG. 6 is a circuit diagram, substantially in block form, of a system modified from that shown in FIG. 1.

### DETAILED DESCRIPTION OF A PREFERRED EMBODIMENT OF THE INVENTION

FIG. 3 is a schematic perspective view of the different layers in a patient's skin. As will be seen, there are a number of layers in the patient's skin. The indications on the left of the figure represent groupings of layers. These groupings of layers are respectively designated as epidermis, dermis and subcutaneous. They include layers designated as stratum corneum, barrier, stratum granulosum, stratum germinativum and papillae.

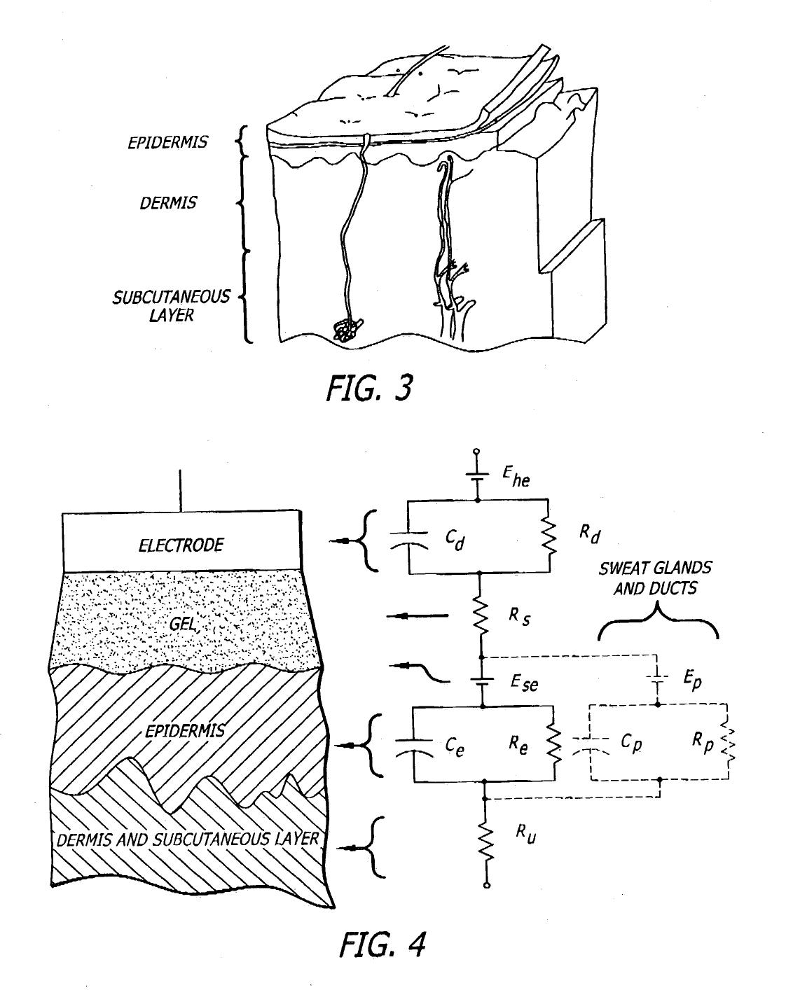

Each of the layers in FIG. 3 has an impedance. This is shown on a schematic basis in FIG. 4, which shows an electrode, a gel, the epidermis layer and a combination of the dermis and subcutaneous layers. In FIG. 4, the gel is shown as being disposed between the electrode and the epidermis to facilitate the coupling of the electrode to the epidermis layer with a minimal impedance.

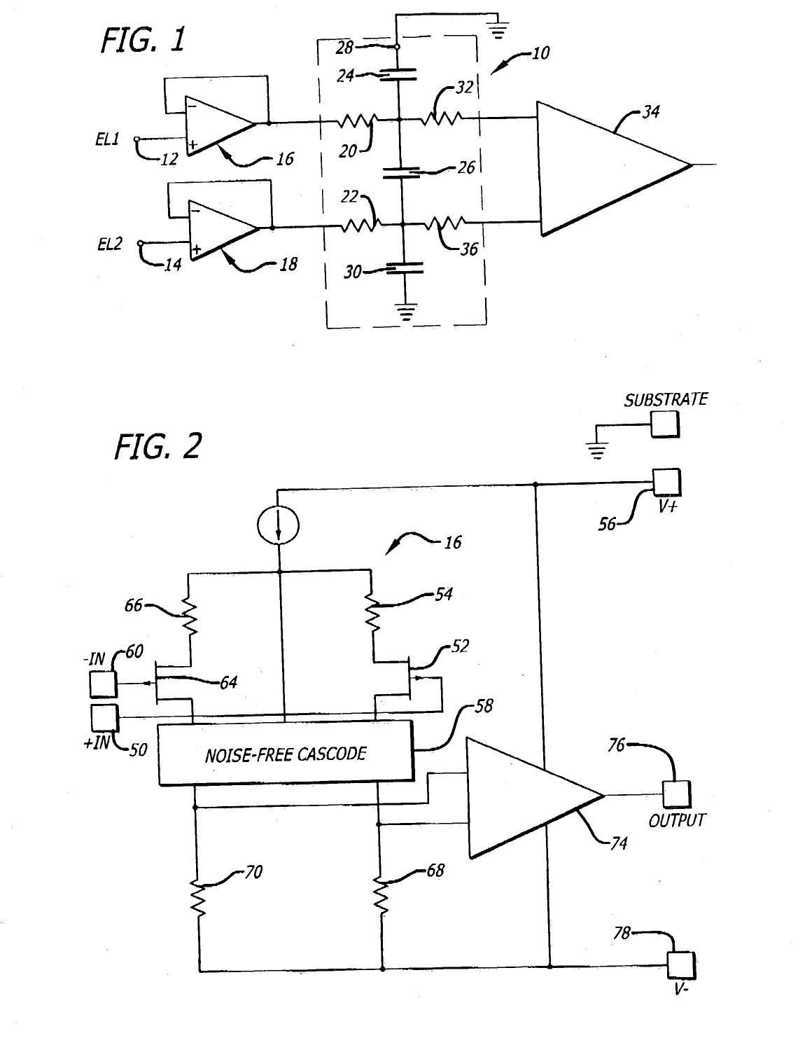

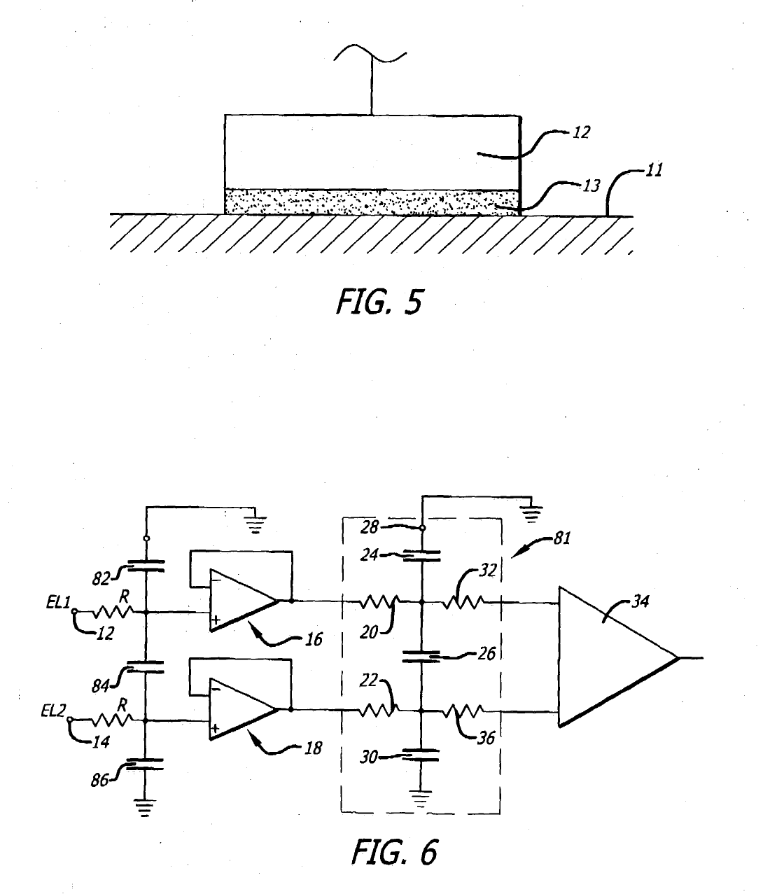

FIG. 5 is a schematic view showing the attachment of an electrode 12 in FIG. 1 to a patient's skin 11 to provide signals for introduction to the amplifier system also shown in FIG. 1. A gel 13 may be disposed between the electrode 10 and the patient's skin 11 to facilitate the attachment of the electrode to the patient's skin. Since each of the layers has an impedance, the collective impedance of the patient's skin is progressively reduced when the successive layers are removed. With all of the layers in place on the patient's skin, the impedance of the patient's skin may be in the order of approximately two hundred thousand (200,000) ohms. However, the amplifier system in FIG. 1 is constructed to operate satisfactorily even when successive layers are not removed from the patient's skin 11 and the electrode 10 is attached to the outside layer.

FIG. 1 is a circuit diagram, primarily in block form, of an amplifier system, generally indicated at 10, constituting a preferred embodiment of the invention. The amplifier system 10 includes a pair of electrodes 12 and 14 each of which is suitably attached to skin at a selective position on the patient's body. The electrodes 12 and 14 preferably have an identical construction. The electrode 12 is positioned at a selective position on the skin of the patient's body to produce signals related to the functioning characteristics of an organ in the patient's body. The organ may illustratively be the patient's heart, brain or the patient's stomach or intestines. The electrode 14 is positioned on the skin of the patient's body at a position displaced from the selective position to provide reference signals. The difference between the signals at the electrodes 12 and 14 represents the functioning characteristics of the selected one of the patient's organs.

The signals on the electrode 12 are introduced to an input terminal of an amplifier generally indicated at 16. The amplifier 16 also has a second input terminal which is connected to the output of the amplifier. In this way, the amplifier acts as a unity gain. The amplifier 16 may be purchased as an OPA 129 amplifier from the Burr-Brown Company which is located in Phoenix, Ariz. In like manner, the signals from the electrode 14 are introduced to an input terminal of an amplifier, generally indicated at 18, which may be identical to the amplifier 16. The amplifier 18 has an input terminal which is connected to the output terminal of the amplifier to have the amplifier act as a unity gain.

Resistors 20 and 22 respectively extend from the output terminals of the amplifiers 16 and 18. The resistor 20 is connected to first terminals of capacitors 24 and 26. The second terminal of the capacitor 24 receives a reference potential such as ground. A connection is made from the resistor 22 to the second terminal of the capacitor 26 and to a first terminal of a capacitor 30, the second terminal of which is provided with the reference potential such as ground. The resistors 20 and 22 may have equal values and the capacitors 24 and 30 may also have equal values.

One terminal of a resistor 32 is connected to the terminal common to the capacitors 24 and 26. The other terminal of the resistor 32 has a common connection with a first input terminal of an amplifier 34. In like manner, a resistor 36 having a value equal to that of the resistor 32 is connected at one end to the terminal common to the capacitors 26 and 30 and at the other end to a second input terminal of the amplifier 34.

Since the amplifiers 16 and 18 have identical constructions, they operate to provide signals which represent the difference between the signals on the electrodes 12 and 14. This indicates the functioning of the patient's organ which is being determined by the amplifier system 30. Although the electrodes 12 and 14 are displaced from each other on the skin of the patient's body, they tend to receive the same noise signals. As a result, the difference between the signals on the output terminals of the amplifiers 16 and 18 does not include any noise.

The electrodes 12 and 14 respectively provide an impedance of approximately 10⁶ ohms to the amplifiers 16 and 18. Each of the amplifiers 16 and 18 respectively provides an input impedance of approximately 10¹⁵ ohms. This impedance is so large that it may be considered to approach infinity. This causes each of the amplifiers 16 and 18 to operate as if it has an open circuit at its input. The output impedance of each of the amplifiers 16 and 18 is approximately 50 ohms to 75 ohms.

Because of the effective open circuit at the input of each of the amplifiers 16 and 18, the output signal from each of the amplifiers 16 and 18 corresponds to the input signal to the amplifiers and does not have any less magnitude compared to the amplitude of the input signal to the amplifier. This is important in view of the production of signals in the microvolt or millivolt region in the electrodes 12 and 14.

The capacitors 24, 26 and 30 and the resistors 20 and 22 provide a low-pass filter and a differential circuit and operate to eliminate the noise on the electrodes 12 and 14. The capacitors 24, 26 and 30 also operate to provide signals which eliminate the commonality between the signals in the electrodes 12 and 14 so that only the signals individual to the functionality being determined relative to the selected organ in the patient's body remain. The capacitors 24, 26 and 30 operate as a low pass filter and pass signals in a range to approximately one kilohertz (1 KHz). The signals having a frequency above approximately one kilohertz (1 KHz) are atentuated.

The amplifiers 16 and 18 are identical. Because of this, a description of the construction and operation of the amplifier 16 will apply equally as well to the amplifier 18. The amplifier 16 is shown in detail in FIG. 2. It is manufactured and sold by Burr-Brown in Phoenix, Ariz. and is designated by Burr-Brown as the OPA 129 amplifier.

As shown in FIG. 2, the amplifier 16 includes an input terminal 50 which receives the signals at the electrode 12 and introduces these signals to the gate of a transistor 52. The source of the transmitter 52 receives a positive voltage from a terminal 56 through a resistor 54. The emitter of the transistor 52 is common with an input terminal in a noise free cascode 58.

Another terminal 60 receives the signals on the electrode 14 and introduces those signals to a gate of a transistor 64. A connection is made from the source of the transistor 64 to one terminal of a resistor 66, the other terminal of which receives the voltage from the terminal 56. The emitter of the transistor 64 is common with an input terminal in the noise-free cascode 58. The resistor 66 has a value equal to that of the resistor 54 and the transistors 52 and 64 have identical characteristics.

First terminals of resistors 68 and 70 having equal values are respectively connected to output terminals in the noise-free cascode 58 and input terminals of an amplifier 74. The amplifier 74 provides an output at a terminal 76. The output from the terminal 76 is introduced to the input terminal 60. The amplifier receives the positive voltage on the terminal 56 and a negative voltage on a terminal 78. Connections are made to the terminal 78 from the second terminals of the resistors 68 and 70.

The transistors 52 and 64 operate on a differential basis to provide an input impedance of approximately 10¹⁵ ohms between the gates of the transistors. The output impedance from the amplifier 16 is approximately fifty (50) ohms to seventy-five (75) ohms. Because of the high input impedance of approximately 10¹⁵ ohms, the amplifier 16 provides an input impedance approaching infinity. This causes the amplifier 16 to provide the equivalent of an open circuit at its input. This causes substantially all of the voltage applied to the input terminal 50 to be provided at the output of the amplifier 16. This is facilitated by the low impedance of approximately fifty ohms (50 ohms) to seventy-five (75) ohms at the output of the amplifier 12. This voltage has characteristics corresponding to the characteristics of the voltage at the electrode 12.

The output signals from the amplifiers 16 and 18 are respectively introduced to the terminal common to the capacitors 24 and 26 and to the terminal common to the capacitors 26 and 30. The capacitors 24, 26 and 30 operate as a low-pass filter to remove noise and to provide an output signal representing the difference between the signals on the electrodes 12 and 14.

The capacitors 24, 26 and 30 correspond to the capacitors C2, C1 and C3 in a low pass filter 76 in application Ser. No. 10/293,105 (attorney's file RECOM-61830) filed on Nov. 13, 2002 in the USPTO and assigned of record to the assignee of record in this application. The capacitors C2, C1 and C3 in application Ser. No. 10/293,105 are included in the low pass filter 76 in FIG. 8-1 (also shown in FIG. 4) of such application. The low pass filter 76 eliminates noise and passes signals through a frequency range to approximately one kilohertz (1 KHz). If any further information may be needed concerning the construction and operation of the low pass filter, reference may be made to co-pending application Ser. No. 10/293,105 to obtain this information.

FIG. 6 shows a preferred embodiment, generally indicated at 81, constituting a modification of the amplifier system 10 shown in FIG. 1. It is identical to the amplifier system 10 shown in FIG. 1 except that it includes capacitors 82, 84 and 86 respectively corresponding to the capacitors 24, 26 and 30 also shown in FIG. 1. The capacitors 82, 84 and 86 are connected as a low pass filter at the inputs of the amplifiers 16 and 18. Like the capacitors 24, 26 and 30, the capacitors 82, 84 and 86 operate as a low pass filter. The addition of the capacitors 82, 84 and 86 provides certain advantages. For example, it assures that no noise passes through the amplifier system 80. Furthermore, it assures that the amplifier system 80 provides stable output signals even when the amplifier system is included in an ambulatory system for measuring the heart characteristics of a patient.

Although this invention has been disclosed and illustrated with reference to particular embodiments, the principles involved are susceptible for use in numerous other embodiments which will be apparent to persons of ordinary skill in the art. The invention is, therefore, to be limited only as indicated by the scope of the appended claims.

1. In combination for providing at selective positions on a patient's skin signals representing the patient's parameters at these positions,
an electrode constructed to be attached to the patient's skin at the selective positions to provide signals indicative of the parameters on the patient's body at the selective positions,
an amplifier having an input terminal with an impedance approaching infinity and providing at the output terminal signals corresponding to the signals from the electrode, and
an output stage connected to the amplifier and constructed to reject noise and to pass signals at frequencies below a particular value.

2. In a combination as set forth in claim 1 wherein
a common mode rejection is provided to the signals from the electrode to eliminate noise from the signals from the electrode before the introduction of the signals to the amplifier.

3. In a combination as set forth in claim 1 wherein
the input impedance of the amplifier is approximately 10¹⁵ ohms.

4. In a combination as set forth in claim 1 wherein
the impedance of the patient's skin is in a range to approximately 200 kilohms and wherein the electrode is attached to the patient's skin.

5. In a combination as set forth in claim 2 wherein
the input impedance of the amplifier is approximately 10¹⁵ ohms, and wherein
the impedance of the patient's skin is in a range to approximately 200 kilohms, and wherein
the electrode is attached to the patient's skin.

6. In a combination for providing signal at selective positions on a patient's skin of the patient's parameters at the selective positions,
an electrode constructed to be applied to the selective positions of the patient's skin to provide a signal representative of the patient's parameters at these selective positions,
an amplifier connected to the electrode to amplify the signals at the electrode, and
a low pass filter connected to the amplifier to provide an output in which noise is eliminated and signals in a particular frequency range are passed by the low pass filter,
the amplifier having characteristics of providing a high input impedance and a low output impedance.

7. In a combination as set forth in claim 6 wherein
the amplifier constitutes a differential amplifier for eliminating noise from the signals provided by the electrode.

8. In a combination as set forth in claim 6 wherein
the amplifier includes a differential stage for eliminating noise from the signals provided by the electrode.

9. In a combination as set forth in claim 6 wherein
the amplifier provides an input impedance approaching infinity.

10. In a combination as set forth in claim 6 wherein
the amplifier and the high pass filter are disposed on a printed circuit board and the amplifier is isolated electrically from the high pass filter on the printed circuit board.

11. In a combination as set forth in claim 9 wherein
the high pass filter limits the amplitude of the output from the high pass filter to facilitate the operation of the amplifier in processing the signals and wherein
the amplifier has a low output impedance.

12. In a combination as set forth in claim 6 wherein
the amplifier provides an input impedance approaching infinity, and wherein
the amplifier and the high pass filter are disposed on a printed circuit board and the amplifier is isolated electrically from the high pass filter on the printed circuit board, and wherein
the high pass filter limits the amplitude of the output from the high pass filter to facilitate the operation of the amplifier in processing the signals and wherein
the amplifier has a low output impedance.

13. In combination for providing at selective positions on a patient's skin signals representing the patient's parameters at these positions,
a first electrode constructed to be attached to the patient's skin at the selective positions to provide signals representing the patient's parameters at these positions,
a second electrode constructed to be attached to the patient's skin at positions different from the selective positions to provide reference signals,
amplifiers connected to the first and second electrodes and having properties of providing a high input impedance approaching infinity and having a low output impedance, and
a high pass filter connected to the amplifiers for eliminating noise and for passing signals at relatively high frequencies.

14. In a combination as set forth in claim 13 wherein
the amplifiers are constructed to obtain the difference between the signals on the first and second electrodes.

15. In a combination as set forth in claim 13 wherein
the amplifiers provide a differential relationship for eliminating noise.

16. In a combination as set forth in claim 13 wherein
the combination of the patient's skin and each individual one of the electrodes has an impedance to approximately 200 kilohms and the amplifier has an input impedance of approximately 10¹⁵ ohms.

17. In a combination as set forth in claim 13 wherein
the combination of the patient's skin and each individual one of the electrodes has an impedance to approximately 200 kilohms and the amplifier has an input impedance of approximately 10¹⁵ ohms.

18. In a combination as set forth in claim 13 wherein
each of the amplifiers has an output impedance of approximately fifty (50) ohms to seventy-five (75) ohms.

19. In a combination as set forth in claim 13 wherein
the amplifiers are constructed to obtain the difference between the signals on the first and second electrodes and wherein
the amplifiers provide a differential relationship for eliminating noise.

20. In a combination as set forth in claim 19 wherein
the combination of the patient's skin and each individual one of the electrodes has an impedance to approximately 200 kilohms and the amplifier has an input impedance of approximately 10¹⁵ ohms
each of the amplifiers has an output impedance of approximately fifty (50) ohms.

21. In combination for providing at selective positions on a patient's skin first signals representing the patient's parameters at these positions,
a first electrode coupled to the patient's skin at one of the selective positions for producing first signals representing the patient's parameter at this position,
a second electrode coupled to the patient's skin at a position other than the selective position for producing reference signals,
a first amplifier coupled to the first electrode for amplifying the first signals, the first amplifier having an input impedance approaching infinity, and
a second amplifier coupled to the second electrode for amplifying the second signals, the second amplifier having an input impedance approaching infinity, and
a differential circuit connected to the first and second amplifiers to eliminate noise and to produce an output signal representing the difference between the first and second signals.

22. In a combination as set forth in claim 21 wherein
the first and second amplifiers have substantially identical characteristics.

23. In a combination as set forth in claim 21 wherein
each of the amplifiers has an input impedance of approximately 10¹⁵ ohms and having an output impedance of approximately 50 ohms to 75 ohms.

24. In a combination as set forth in claim 27 wherein the first and second amplifiers have substantially identical characteristics.

25. In combination for providing at selective positions on a patient's skin first signals representing the patient's parameters at these positions,
an electrode coupled to the patient's skin at one of the selective positions for producing second signals representing the patient's parameters at this position, and
an amplifier connected to the first electrode for amplifying the signals from the electrode, the amplifier having an input impedance approaching infinity.

26. In a combination as set forth in claim 25 wherein
the amplifier has an input impedance of approximately 10¹⁵ ohms.

27. In a combination as set forth in claim 25 wherein
the amplifier has an output impedance considerably less than the input impedance of the amplifier.

28. In a combination as set forth in claim 26 wherein
the amplifier has an output impedance of approximately 50 ohms to 75 ohms.

29. In a combination as set forth in claim 26,
a low pass filter coupled to the output of the amplifier to receive the signals from the amplifier, and
a printed circuit board for holding the amplifier and the low pass filter with the amplifier in physically and electrically displaced relationship to the low pass filter.

30. In a combination as set forth in claim 29,
a second low pass filter connected between the electrode and the input to the amplifier to pass signals below a particular frequency.

31. In a combination as set forth in claim 29 wherein
the differential circuit is a first differential circuit and is connected to the outputs of the amplifiers to operate as a low pass filter for passing signals below a particular frequency and to eliminate noise and wherein
a second differential circuit is connected between the electrode and the amplifiers to operate as a low pass filter for passing signals below the particular frequency and to eliminate noise.

32. In a combination as set forth in claim 1 wherein
the amplifier has an input and an output and wherein
the output stage is connected to the output of the amplifier and wherein
a second stage is connected between the electrode and the input of the amplifier and is constructed to reject noise and to pass signals at frequencies below the particular value.

33 In a combination as set forth in claim 6 wherein
the amplifier has an input and an output and wherein
the low pass filter is a first low pass filter and is connected to the output of the amplifier to provide an output in which noise is eliminated and signals in the particular frequency range are passed by the low pass filter and wherein
a second low pass filter is connected between the electrode and the input of the amplifier to eliminate noise and to pass signals in the particular frequency range.

34. In a combination as set forth in claim 6 wherein
the first low pass filter operates on a differential basis and wherein
the second low pass filter operates on a differential basis.
